(19) United States
(12) Patent Application Publication
(10) Pub. No.: US 2025/0248917 A1
(43) Pub. Date: Aug. 7, 2025

(54) Multifunctional Skincare Compositions for Anti-Aging, Hydration, Barrier Repair, and Sensitivity Relief

(71) Applicant: yong hu, Maple Valley, WA (US)

(72) Inventor: yong hu, Maple Valley, WA (US)

(21) Appl. No.: 19/094,813

(22) Filed: Mar. 29, 2025

(51) Int. Cl. A61K 8/67; A61K 8/31; A61K 8/49; A61K 8/64; A61K 8/68; A61K 8/73; A61K 8/99; A61Q 19/00; A61Q 19/08

(52) U.S. Cl. CPC A61K 8/675; A61K 8/31; A61K 8/4953; A61K 8/64; A61K 8/68; A61K 8/735; A61K 8/99; A61Q 19/005; A61Q 19/08; A61K 2800/592

(57) ABSTRACT

The invention provides a multifunctional skincare composition set designed for synergistic performance in hydration, anti-aging, barrier repair, and sensitivity relief. Each product in the three-part system—comprising a toner, emulsion, and cream—utilizes a phase-based formulation strategy for optimal ingredient stability. Key actives include niacinamide, multimolecular hyaluronic acid, ceramide complex, bioferments, peptides, and plant stem cell extracts. The formulation addresses common skincare concerns through layered delivery of hydrating, lipid-replenishing, and regenerative ingredients.

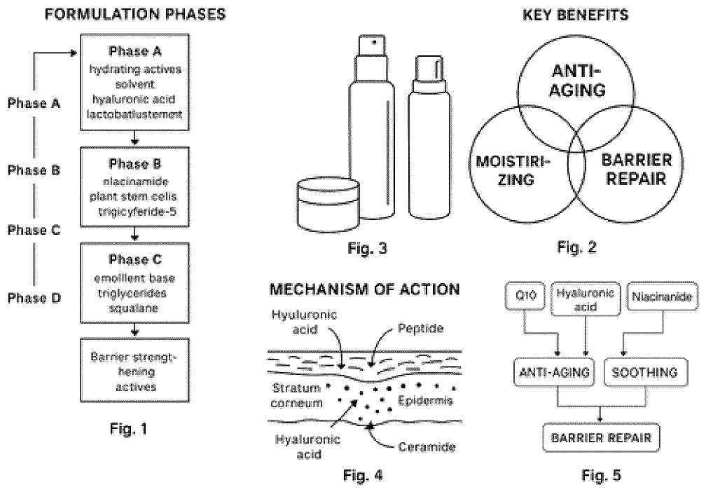

### 1. FIELD OF THE INVENTION

This invention pertains to advanced skincare compositions, specifically a synergistic set of multifunctional cosmetic formulations designed to deliver enhanced moisturization, anti-aging benefits, skin barrier restoration, and sensitivity relief for diverse skin types, including sensitive and post-procedure skin.

### 2. BACKGROUND OF THE INVENTION

Modern skincare consumers seek multifunctional products that combine intensive hydration, anti-aging efficacy, skin barrier repair, and sensitivity alleviation. Existing formulations often fail to deliver stable, synergistic combinations of actives such as recombinant collagen, peptides, ceramides, and bioferments due to compatibility and stability challenges. Traditional single-purpose products lack the comprehensive performance required for sensitive, dry, or aging skin.

This invention overcomes these limitations by introducing a stable, phased-delivery skincare system optimized for efficacy and compatibility across a three-product regimen.

### 3. SUMMARY OF THE INVENTION

Disclosed herein is a skincare system comprising three complementary products:
Hydra Repair Essence Toner
Hydra Recovery Emulsion
Hydra Fortifying Cream

Each product integrates a precise blend of active ingredients categorized by function (moisturization, anti-aging, barrier repair, and sensitivity relief). These actives are incorporated through a phased manufacturing process to ensure stability, bioavailability, and synergistic performance. Key components include recombinant human collagen, multi-molecular hyaluronic acid, peptide complexes, ceramide-lipid systems, and biofermented extracts, formulated to address hydration, fine lines, barrier integrity, and sensitivity.

### 4. DETAILED DESCRIPTION OF THE PREFERRED EMBODIMENTS

The skincare system comprises three products, each formulated with ingredients from the following functional categories:

### A. Core Functional Actives

Recombinant Human Collagen: 0.3-0.5% (promotes epidermal repair and barrier restoration)
Niacinamide: 2-5% (brightens skin, reduces fine lines, enhances barrier function)
Sodium Hyaluronate (Multi-Molecular: High, Medium, Low): 0.1-0.4% (provides layered hydration)
Crosslinked Sodium Hyaluronate: 0.2-0.4% (long-term hydration and skin plumpness)
Polyglutamic Acid (PGA): 0.3-0.5% (long-lasting moisture retention, mimics NMF)
Ceramide Complex (NP/AP/EOP): 1.5-4% (rebuilds lipid barrier)
Cholesterol and Plant Sterols: 0.5-1% (stabilizes lipid matrix, mimics skin lipids)
Peptides (Tripeptide-1, Hexapeptide-8, Acetyl Octapeptide-3): 0.03-0.05% each (stimulates collagen synthesis, reduces wrinkles)
Glucan: 0.3-1% (soothes irritation, strengthens barrier)
Centola Asiatica Extract: 0.2-0.3% (anti-inflammatory, sensitivity relief)
Coenzyme Q10 (Ubiquinone): 0.05-0.1% (antioxidant, supports cellular renewal)
Trehalose: 0.2-0.3% (moisture retention, antioxidant protection)
Fermented Filtrate (e.g., Lactobacillus/Soy): 0.5-2% (enhances skin microbiome, hydration)

### B. Supportive Matrix

Panthenol: 0.5-2% (deep hydration, barrier repair)
Glycerin: 2-3% (immediate hydration, humectant)
Betaine: 1-2% (moisture retention, soothing)
Squalane: 5-10% (emollient, barrier enhancement)
Jojoba Oil: 1-2% (nourishing, non-comedogenic)
Caprylic/Capric Triglyceride: 3-4% (lightweight emollient, improves texture)
Dimethicone: 0.5-1% (occlusive, smooths skin)
Emulsifiers (e.g., PEG-100 Stearate, Glyceryl Stearate, Cetearyl Alcohol): 15% (stabilizes formulations)

### C. Preservation and Stabilization

Phenoxyethanol+Ethylhexylglycerin: 0.7-0.8% (mild preservative system, sensitive skin-friendly)
Disodium EDTA: 0.01-0.3% (chelating agent, enhances stability)
Triethanolamine/Citric Acid: q.s. to pH 5.5-6.5 (optimizes skin compatibility)

### D. Phase Addition Strategy

To ensure ingredient stability and efficacy:
Phase A (Water-Soluble Base): Glycerin, polyglutamic acid, trehalose, panthenol, recombinant collagen, and-glucan added at 35-45° C.
Phase B (Oil Phase): Squalane, ceramides, cholesterol, jojoba oil, and emulsifiers blended at 70-75° C.
Phase C (Heat-Sensitive Actives): Peptides, Coenzyme Q10, and Centella Asiatica extract incorporated at 45° C. to preserve bioactivity.
Phase D (Final Stabilization): Preservatives and pH adjusters added at room temperature, followed by sterile filtration (0.22 μm) and aseptic packaging.

Product-Specific Compositions:
1. Deep Hydration Essence Toner: Emphasizes recombinant collagen (0.5%), polyglutamic acid (0.5%), multi-molecular hyaluronic acid (0.4%), and-glucan (1%) for 72-hour hydration and sensitivity relief.
2. Firming Repair Emulsion: Focuses on peptides (Tripeptide-1, Hexapeptide-8, Acetyl Octapeptide-3 at 0.03-0.05% each), ceramide complex (3%), and niacinamide (3%) for wrinkle reduction and barrier repair.
3. Renewal Fortifying Cream: Prioritizes ceramide complex (4%), cholesterol (1%), crosslinked hyaluronic acid (0.4%), and Coenzyme Q10 (0.1%) for deep barrier restoration and nighttime rejuvenation.

### 5. ADVANTAGES

Synergistic Efficacy: Combines medical-grade repair (recombinant collagen, ceramides) with advanced anti-aging (peptides, Coenzyme Q10) and long-lasting hydration (polyglutamic acid, hyaluronic acid).
Sensitive Skin Optimization: pH-balanced (5.5-6.5), alcohol-free, fragrance-free, and cruelty-free, validated for sensitive skin via 72-hour patch testing.
Stability and Bioactivity: Phased manufacturing preserves fragile actives, ensuring consistent performance.
Comprehensive Care: Three-product system addresses daily and specialized skincare needs, surpassing single-function formulations.

1. A cosmetic composition comprising a synergistic combination of niacinamide, peptides (palmitoyl tripeptide-5 and/or acetyl tetrapeptide-11), ceramide complex, ectoine, fermented extract, and squalane.

2. The composition of claim 1, wherein said actives are added in separate thermal phases to ensure bioactivity and product stability.

3. The composition of claim 1, formulated for use as a three-product skincare set consisting of a toner, emulsion, and cream, wherein each product specializes in delivering hydration, barrier enhancement, and anti-aging benefits.

4. The composition of claim 1, wherein at least one plant-based stem cell extract is included in concentrations ranging from 0.1 to 0.5%.

5. The composition of claim 1, wherein the peptide content is present at a total amount between 0.1 to 0.5%, in combination with ceramide-cholesterol systems for barrier restoration.
